(19) United States
(12) Patent Application Publication
(10) Pub. No.: US 2025/0327789 A1
(43) Pub. Date: Oct. 23, 2025

(54) BULK VOLUME MEASUREMENT PREDICTION VIA MULTI-TECHNIQUE SURFACE ANALYSIS

(71) Applicant: SAUDI ARABIAN OIL COMPANY, Dhahran (SA)

(72) Inventors: Mustafa HAKIMUDDIN, Dhahran (SA); Sultan M. ENEZI, Dhahran (SA)

(73) Assignee: SAUDI ARABIAN OIL COMPANY, Dhahran (SA)

(21) Appl. No.: 18/642,680

(22) Filed: Apr. 22, 2024

(51) Int. Cl. E21B 47/002; G01N 33/24

(52) U.S. Cl. CPC G01N 33/241

(57) ABSTRACT

A system for generating a predictive model using an input specimen includes a bulk volume prediction engine to generate the predictive model using one or more scans of the input specimen. The bulk volume prediction engine includes a training module to train a neural network on training data including scans of training input specimens and known parameters for training input specimens, a neural network module to determine bulk volume data and surface pore volume calculations for a body of interest using the scans of the input specimen as an input to a trained neural network, and a decision module to test results of the trained neural network compared against known training parameters.

### FIELD OF THE DISCLOSURE

The present disclosure relates generally to bulk volume measurements, and, more particularly, to systems and methods for prediction of bulk volume parameters using data from surface analysis.

### BACKGROUND OF THE DISCLOSURE

Cylindrical rock samples are commonly extracted from hydrocarbon reservoirs for use in data collection and analysis. The data collected from these rock samples can be utilized in calibration of further data collection and in assessment of a potential of a hydrocarbon reservoir. An operator can determine an estimated hydrocarbon volume, production capacity, and further operational parameters for the hydrocarbon reservoir. The estimated potential of the hydrocarbon reservoir can depend upon a porosity value derived from the cylindrical rock sample obtained therefrom. The porosity value can define a capacity of the hydrocarbon reservoir to hold fluids therein, such that increased porosity can suggest a greater hydrocarbon capacity. As such, an accurate determination of porosity within a hydrocarbon reservoir can be required for estimation of capacity in a hydrocarbon reservoir.

Several techniques can be employed in measuring porosity of a rock sample for extrapolation to the entirety of the hydrocarbon reservoir. Some caliper methods can be utilized in cylindrical rock samples; however, any irregularities or imperfections in the cylindrical shape can drastically increase inaccuracies using the caliper methods. Further methods include fluid displacement testing to determine bulk volume when submerged in a fluid. One such method can use the Archimedes principle and a non-wetting fluid, such as mercury, for determination of porosity and bulk volume. The use of mercury can lead to negative environmental and health effects, and can further render the rock sample useless in further testing as the sample can be destroyed or plugged during testing. Further fluid displacement testing can use wetting fluids which can enter the rock sample and affect the bulk volume measurements. Further, these subsurface reservoir rock samples can be inundated with surface irregularities such as vugs, fractures, chipped edges, open pores, and other surface irregularities related to plugging and surfacing processes. These irregularities can create errors in bulk volume measurements that can lead to incorrect values for porosity of the reservoir, which is the measure of a reservoir's capacity to hold fluids.

As such, the use of conventional techniques can lead to incorrect measurements and damage to the rock sample. Incorrect measurement can further lead to under-estimation of a reservoir's capacity in the range of 5% to 25%. For example, for a hydrocarbon reservoir estimated to have one billion barrels of oil with a 50% production capacity will equate to a reservoir capacity of $90 billion with revenue generation capacity of $45 billion, assuming an average price per barrel of $90. The under-estimation of these reservoirs due to conventional techniques can lead to losses anywhere between $4.5 billion to $22.5 billion in reserve capacity and anywhere between $2.25 billion to $11.25 billion in revenue generation capacity. The under-estimation can, in-turn, lead to the abandonment of reservoirs that are estimated to be on the lower end of overall reserve capacity, as cost-benefit analysis may render these reservoirs as unprofitable. As such, under-estimation can lead to lost opportunities from revenue prospecting as well as premature and aggressive exploration activities for further hydrocarbon reservoirs. The overall effect of the under-estimation can negatively impact both the financials of the reservoir extraction operator as well as the environment. Further, incorrect bulk volume measurement can also lead to over-estimation of the reservoir's capacity, depending on the source of the measurement error. In these cases, over-estimation can lead to wasted resources and an overall loss from the hydrocarbon field, as investments fail to match production.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a system for generating a predictive model using an input specimen includes a bulk volume prediction engine to generate the predictive model using one or more scans of the input specimen. The bulk volume prediction engine includes a training module to train a neural network on training data including scans of training input specimens and known parameters for training input specimens, a neural network module to determine bulk volume data and surface pore volume calculations for a body of interest using the scans of the input specimen as an input to a trained neural network, and a decision module to test results of the trained neural network compared against known training parameters.

In another embodiment, a computer-implemented method for training a neural network to predict bulk volume data from an input specimen includes performing, via a scanner assembly, one or more scans of an input specimen from a body of interest with known bulk volume parameters, receiving one or more of the known bulk volume parameters for the input specimen or body of interest, and training, via a bulk volume prediction engine, a neural network model to correlate the one or more scans to the known bulk volume parameters through creation of a correlation or refinement of an existing correlation. The computer-implemented method may be performed such that the one or more scans are selected from the group consisting of an optical image scan, a laser reflective scan, an acoustic reflective scan, an x-ray reflective scan, and any combination thereof.

In a further embodiment, a computer-implemented method for predicting bulk volume measurements of a body of interest via a trained neural network includes performing, via a scanner assembly, one or more scans of an input specimen from the body of interest without known bulk volume parameters, calculating, via a predictive model, bulk volume data and/or surface porosity parameters for the body of interest using the one or more scans of the input specimen, and outputting calculated bulk volume data and/or surface porosity parameters for the body of interest to a processing device, wherein the predictive model includes a neural network model trained on scans of test specimens with known parameters.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a block diagram of a system for determining volume and porosity of a bulk volume via an extracted input specimen.

FIG. 2 is an example schematic side view of a scanner assembly for scanning an input specimen, according to at least one embodiment of the present disclosure.

FIG. 3 is an example of a method for training a neural network on scans of an input specimen with known parameters.

FIG. 4 is an example of a method for testing a neural network on scans of an input specimen with known parameters.

FIG. 5 is an example of a method for deploying a trained neural network as a predictive model for an unknown body of interest.

FIG. 6 is a block diagram of a computer system that may be used to implement one or more of the systems or methods described herein in accordance with certain embodiments.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to bulk volume measurements, and, more particularly, to systems and methods for prediction of bulk volume parameters using data from surface analysis. Embodiments herein can include a scanner assembly operable to take a plurality of scans of an input specimen using a variety of scanning technologies including x-rays and acoustic waves. The scanner assembly can autonomously rotate and flip the input specimen to scan each surface of the input specimen. The embodiments disclosed herein can utilize surface molds of the input specimens to further extract surface features, and can further utilize multi-layer scanning procedures to determine surface strength and deformation. The disclosed embodiments can utilize a neural network model trained on known quantities and training data to correlate the obtained scans to one or more bulk volume parameters of a body of interest. In some embodiments, the body of interest can be a hydrocarbon reservoir and the input specimen can be a rock sample extracted therefrom. In further embodiments, however, the body of interest can be an agricultural, pharmaceutical, medical, or construction system which includes bulk parameters relatable to an input specimen.

Through the use of non-invasive and non-destructive scanning methods, higher accuracy predictions of bulk volume measurements can be generated and repeated. Further, through the use of a trained neural network and machine learning, the speed at which the bulk volume parameters can be predicted may be greatly improved. With quality input data and extensive training of the neural network model, machine learning can provide greater accuracy than conventional prediction methods. For embodiments using a hydrocarbon reservoir as a body of interest, the accurate calculation or prediction of porosity and permeability of the reservoir can increase accurate estimates of production capacity and reserves within the reservoir. The neural network can be trained on porosity, permeability, grain shape, grain size, grain spacing, grain density, mineralogy, petrophysical data, well logs, and drilling logs, each of which can be linked to surface scan data generated by scanning the input specimen. As such, with accurate bulk volume parameters, better planning and extraction operations can be performed on the hydrocarbon reservoir without premature killing or abandonment of the well.

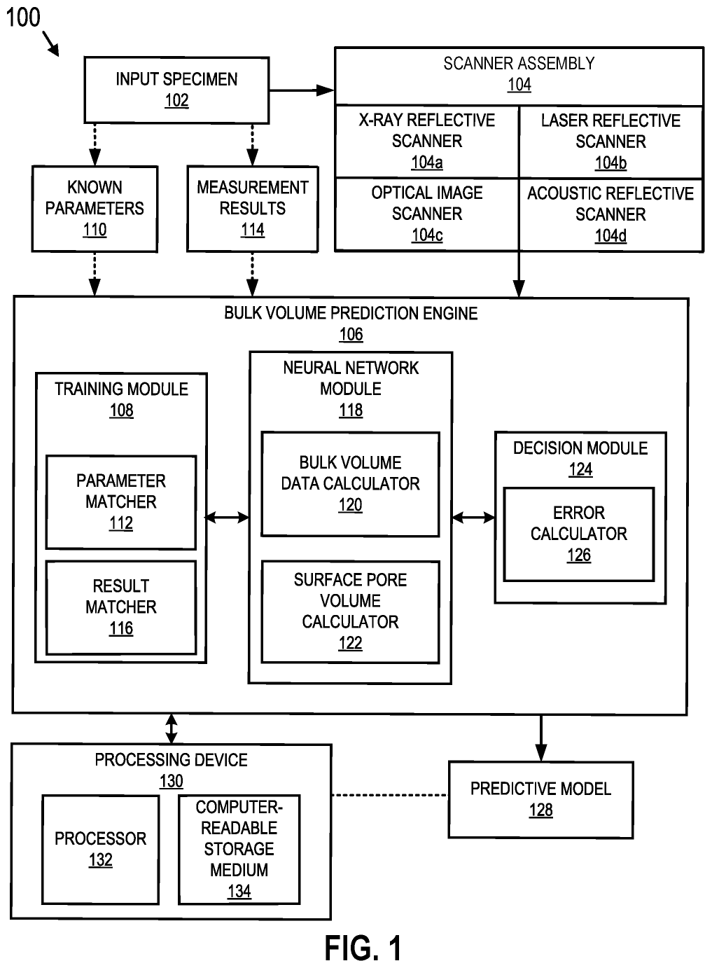

FIG. 1 is a block diagram of a system 100 for determining volume and porosity of a body via an extracted input specimen 102. The input specimen 102 can be extracted from a larger body for prediction of the bulk parameters of the body using a smaller sample. In some embodiments, the body can be a hydrocarbon reservoir and the input specimen 102 can be a cylindrical rock sample extracted therefrom. However, in further embodiments, the body and input specimen 102 can be representative of a medical, agricultural, pharmaceutical, or construction system. The input specimen 102 can be assessed via a scanner assembly 104 which can include a plurality of scanning devices 104a-d for determination of a variety of physical properties and measurements. As discussed above, standard practices may utilize destructive and invasive techniques which can limit reproducibility while introducing error. As such, the scanner assembly 104 can be utilized in both non-invasive and non-destructive prediction techniques.

The scanner assembly 104 can include an x-ray reflective scanner 104a for obtaining surface measurements of the input specimen 102 using x-rays. The x-ray reflective scanner 104a can measure a single surface of the input specimen 102, or can perform a multi-layer examination of the input specimen 102 while investigating a depth, size, and shape of a surface pore into the input specimen 102. The scanner assembly 104 can further include a laser reflective scanner 104b which can employ confocal laser scanning. The laser reflective scanner 104b can utilize laser scanning in analysis and investigation of surface anomalies of the input specimen 102 that can be commonly found in cylindrical rock samples from a hydrocarbon reservoir. The laser reflective scanner 104b can detect clay, wax, asphaltene, salt, and other impurities with differing reflective or absorptive properties from the bulk of the input specimen 102. The difference in reflected laser light can provide additional information about the content of the input specimen 102 via the laser reflective scanner 104b. The scanner assembly 104 can further include an optical image scanner 104c that can obtain images and/or videos of the input specimen 102. The optical image scanner 104c and obtained images can be used with calibrated images for calculation of surface area and other physical measurements of the input specimen 102. The scanner assembly 104 can further include an acoustic reflective scanner 104d that can utilize acoustic waves in assessment of the input specimen 102. The acoustic reflective scanner 104d can provide a depth of the holes present in the input specimen 102, and can determine the depth of the hole as a function of the location within the input specimen 102. The acoustic reflective scanner 104d can therefore aid in obtaining surface measurements as well as understanding the structure of pores, fractures, cracks, and dents within the input specimen 102. Further, the acoustic reflective scanner 104d can detect impurities and surface anomalies, similar to the laser reflective scanner 104b. The acoustic reflective scanner 104d can detect these anomalies based upon the frequency and wave type returned from the input specimen 102 during scanning. Those skilled in the art will readily appreciate that further non-invasive and non-destructive scanners can be included in the scanner assembly 104 without departing from the scope of this disclosure. In some embodiments, selective scanners 104a-d can be selected for scanning of an input specimen 102, while in further embodiments each scanner 104a-d can be utilized in scanning. Following any initial scanning of the input specimen 102, a mold of the input specimen 102 can be created and run through the scanner assembly 104. Through scanning of the mold of the input specimen 102, the scanner assembly 104 can extract further surface characteristics and features from the input specimen 102. In some embodiments, the scanner assembly can perform multi-layer scanning using acoustic and x-ray reflective scanning to visualize surface structural and deformation information for the input specimen 102. In these embodiments, the structural and deformation information can advise mechanical strength predictions and can aid in drilling planning for hydrocarbon reservoirs.

The scanner assembly 104 may collect the plurality of scans performed by each scanner 104a-d, and can provide the plurality of scans to a bulk volume prediction engine 106. The bulk volume prediction engine 106 can be utilized in prediction of the bulk volume and porosity of an input specimen 102 while extrapolating the predictions to the entire body from which the input specimen 102 was obtained. The bulk volume prediction engine 106 can include a training module 108 that is operable to train a neural network on training data. The training data can include the scans received from the scanner assembly 104 as well as one or more known parameters 110 of the input specimen 102 or body of interest. For a rock sample as the input specimen 102, the known parameters 110 can include dimensional data (volume, length, diameter, etc.), geomechanical data (compressibility, linear stress-strain response, etc.), well data (petrophysical data, drilling fluid data, grain information, mineralogy data, etc.), field log data (mineralogical sample values, impurity presence, sonic and gamma ray logging, etc.), and any combination thereof. In further embodiments, however, the known parameters 110 can include data relating to the application of interest, including but not limited to medicinal, pharmaceutical, agricultural, or construction-related applications.

The known parameters 110 can be determined using the aforementioned destructive or invasive techniques, or can be determined from a body of interest during or after operation. The known parameters 110 and the scans from the scanner assembly 104 can be utilized within a parameter matcher 112 of the training module 108. The parameter matcher 112 can train a neural network model on the input scans from the scanner assembly 104 to obtain a desired output of the known parameters 110. The bulk volume prediction engine 106 and the training module 108 can further receive one or more measurement results 114 for training and prediction. The measurement results 114 can include results from both a caliper measurement and a mercury intrusion test using the Archimedes principle. The difference between the caliper and mercury test can be included in the measurement results 114 as a quantification of surface area and porosity. In some embodiments, the measurement results 114 can further include the one or more scans from the scanner assembly 104 of a surface mold created from the input specimen 102. The surface mold can enable surface porosity information related to pores, indentations, cracks, and further imperfections that can be indicative of porosity. The training module 108 can utilize the measurement results 114 in the training of a neural network model to further account for surface pore volume as part of the desired bulk volume measurements. For hydrocarbon reservoirs, the measurement results 114 can further include the geomechanical data of compressibility and linear stress and strain, well log data, laboratory petrophysical data, grain information, and drilling fluid data to determine correlations between these measurement results and the surface scans of the input specimen 102.

In the illustrated embodiment, the parameter matcher 112 and result matcher 116 are shown as parts of the training module 108, however, in further embodiments one or both of the parameter matcher 112 and result matcher 116 can be components of the neural network module 118. The neural network module 118 and the training module 108 can operate in tandem to train and build a neural network model. The neural network module 118 can utilize a combination of convolutional, modular, and/or auto-encoder neural networks for generation of a neural network model capable of bulk volume prediction.

The neural network module 118 can utilize training by the training module 108 for creating a neural network model that can utilizes a bulk volume data calculator 120 for determination of one or more parameters. The bulk volume data calculator 120 can utilize any relationships and correlations determined in the training module 108 to calculate a bulk volume correlated to the scans received from the scanner assembly 104. Patterns, trends, and both geometric and geological features of the input specimen 102 can be connected to known parameters 110 and measurement results 114 to yield a bulk volume data calculator 120 that may predict bulk volume data of a body of interest using the input specimen 102. In petrophysical applications, the neural network module 118 may further utilize the measurement results 114 with any outputs of the scanner assembly 104 for determination of surface porosity and wellbore characteristics corresponding to the input specimen 102. The surface porosity of the input specimen 102 can further indicate porosity throughout a reservoir that may not be accounted for by traditional methods omitting surface examination.

The bulk volume prediction engine 106 can further include a decision module 124 for autonomous or monitored assessment of a neural network model generated via the training module 108 and neural network module 118. The decision module 124 may receive a plurality of known parameters 110 for a test body and input specimen 102. The neural network model, however, may only receive the outputs of the scanner assembly 104, such that the bulk volume data may be predicted. The decision module 124 may further receive the predicted bulk volume measurement of the test body and input specimen 102 from the neural network model, and may compare the predicted values to the known parameters 110. One or more error values can be calculated with the error calculator 126 between the known parameters 110 and the predicted values, such that an accuracy of the neural network model and bulk volume prediction engine 106 can be quantified. After a plurality of tests are performed, an average error can be generated by the error calculator 126 to determine the overall effectiveness of the neural network model and any generated predictions. The decision module 124 can utilize a threshold to determine if the average error falls within acceptable ranges, or whether further training of the neural network model should be performed. In some embodiments, the average error threshold may be about 5%, while in further embodiments the average error threshold may be smaller, or about 1%.

In some embodiments, the bulk volume prediction engine 106 can output a predictive model 128 which is within the desirable average error threshold. The predictive model 128 can be approved within the decision module 124 and can independently receive data from scanner assembly 104 to generate bulk volume and surface porosity calculations. The predictive model 128 can be deployed to operator devices or stored on a cloud-based system for access by multiple users. In some embodiments, however, the predictive model 128 can remain a part of the neural network module 118. In these embodiments, the predictive model 128 can be further trained and improved while in use, and can be used in generation of further predictive models 128 within the bulk volume prediction engine 106.

In some embodiments, the bulk volume prediction engine 106 and/or the scanner assembly 104 can be in communication with a processing device 130. The processing device 130 can include a processor 132 and a computer-readable storage medium 134, and can be in physical, wired communication with the rest of the system 100. The processing device 130 can include any computing device, for example, a desktop computer, a server, a controller, a blade, a mobile phone, a tablet, a laptop, a personal digital assistant (PDA), or other types of portable (or stationary) devices. By way of example, the computer-readable storage medium 134 can be implemented, for example, as a non-transitory computer storage medium, such as volatile memory (e.g., random access memory), non-volatile memory (e.g., a hard disk drive, a solid-state drive, a flash memory, or the like), or a combination thereof. The processor 132 can be implemented, for example, as one or more processor cores. The computer-readable storage medium 134 can store machine-readable instructions (e.g., the bulk volume prediction engine 106) that can be retrieved and executed by the processor 132. Each of the processor 132 and the computer-readable storage medium 134 can be implemented on a similar or a different computing platform. The computing platform could be implemented in a computing cloud and thus on a cloud computing architecture. In such a situation, features of the computing platform could be representative of a single instance of hardware or multiple instances of hardware executing across the multiple of instances (e.g., distributed) of hardware (e.g., computers, routers, memory, processors, or a combination thereof). Alternatively, the computing platform could be implemented on a single dedicated server or workstation.

In further embodiments, the processing device 130 can be in wireless communication with the rest of the system 100, and can be locally stored, or accessed as a cloud device over the internet. In some embodiments, the processing device 130 can include the bulk volume prediction engine 106, such that the predictive model 128 can be generated and utilized within the processing device 130. The processing device 130 can accordingly be utilized by field petrophysics, lab petrophysics, reservoir management, and reservoir simulation departments for a variety of purposes. The predictive model 128 can enable corrections to bulk volume, porosity, permeability, mineralogy, and the presence of impurities or anomalies such as clay, was, asphaltene, salt, and other non-rock elements. The predictive model 128 can further enable corrections to well logs and characterization of rock properties for the sampled section and corrections to laboratory test data to ensure a unified analysis is performed.

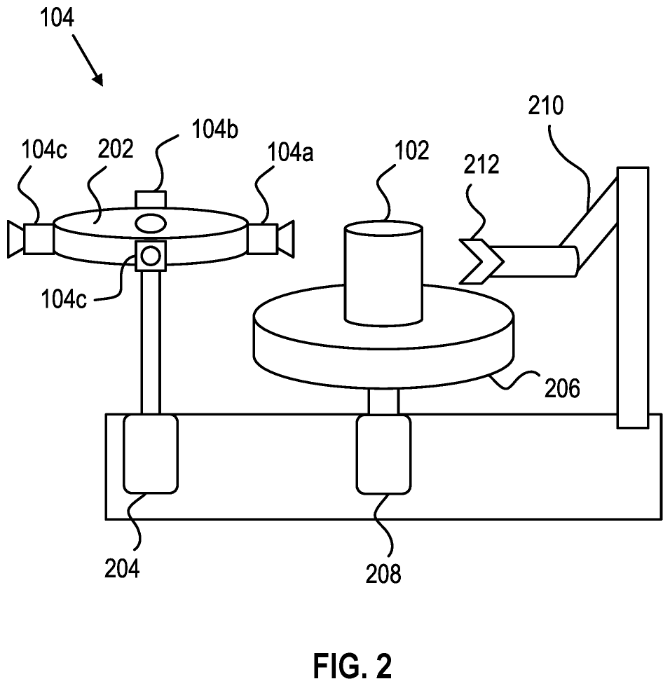

FIG. 2 is an example schematic side view of a scanner assembly 104 for scanning an input specimen 102, according to at least one embodiment of the present disclosure. In some embodiments, the scanner assembly 104 can include each of the scanners 104a-d mounted on a structure 202. In these embodiments, the scanners 104a-d can be mated to the structure 202, which can rotate to place each scanner 104a-d in alignment with the input specimen 102 during operation. In the illustrated embodiment, the structure 202 is a horizontal ring-shaped body on which each scanner 104a-d can be perpendicularly mounted. The structure 202 can be further mated to a scanner motor 204 which can rotate the structure 202 about the central axis of the ring-shaped body. Those skilled in the art will readily appreciate that the structure 202 can be shaped, oriented, and controlled in any manner that positions the scanners 104a-d in alignment with the input specimen 102 without departing from the scope of this disclosure.

The scanner assembly 104 can further include a specimen base 206 on which the input specimen 102 can be placed. The specimen base 206 can be a rotatable platform that utilizes bearings, or any other low-friction mechanism, to enable rotation of the input specimen 102 during scanning. In some embodiments, the specimen base 206 can include a specimen motor 208 that may power rotation of the specimen base 206 during operation. The scanner assembly 104 can further include a flipper arm 210 at or near the specimen base 206. The flipper arm 210 can include a grip 212 operable to grasp the input specimen 102 during scanning. The flipper arm 210 and grip 212 can rotate to flip the input specimen 102 such that a side of the input specimen 102 that was previously disposed on the specimen base 206 can be exposed to the scanners 104a-d. Through the series orientation of the scanners 104a-d, the rotatability of the specimen base 206, and the flipping of the flipper arm 210 can enable a full, autonomous scan of the input specimen 102 by each scanner 104a-d.

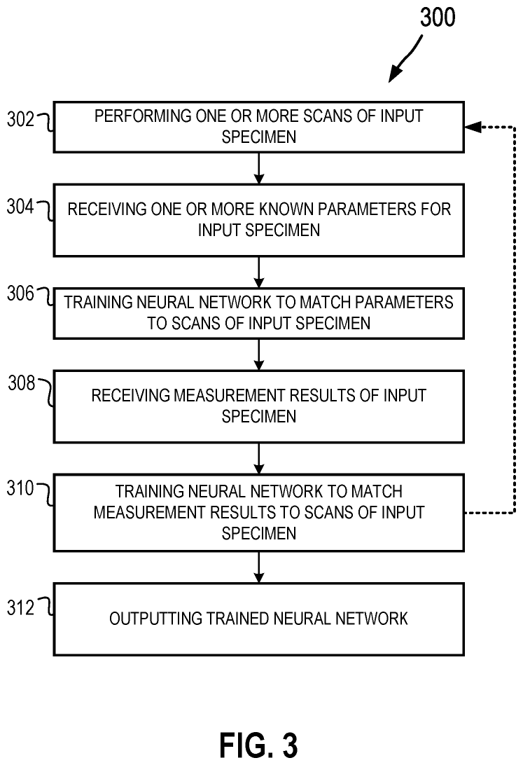

In view of the structural and functional features described above, example methods will be better appreciated with reference to FIGS. 3-5. While, for purposes of simplicity of explanation, the example methods of FIGS. 3-5 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the methods, and conversely, some actions may be performed that are omitted from the description.

FIG. 3 is an example of a method 300 for training a neural network on scans of an input specimen with known parameters. The method 300 can be implemented by the system 100 and the scanner assembly 104, as shown in FIGS. 1-2. Thus, reference can be made to the example of FIGS. 1-2 in the example of FIG. 3. The method 300 can begin at 302 with performing one or more scans of an input specimen (e.g., the input specimen 102). The one or more scans performed at 302 can be performed by a scanner assembly (e.g., the scanner assembly 104) and can include an x-ray reflective scan, a laser reflective scan, an optical image scan, an acoustic reflective scan, or any combination thereof (e.g., performed by the scanners 104a-d). The scans at 402 can be performed on each surface of an input specimen, and can further include one or more scans of a surface mold for the input specimen. The surface mold of the input specimen can be scanned for extraction of surface features to aid in determining surface porosity of the input specimen. Further, in some embodiments, the one or more scans can include multi-layer scans using an x-ray scanner and an acoustic scanner for understanding of surface deformation and failure mechanics.

The method 300 can further include receiving one or more known parameters (e.g., the known parameters 110) for the input specimen at 304. For hydrocarbon reservoir applications, the one or more known parameters can include, but are not limited to, bulk volume, length, diameter, porosity, surface pore volume, geomechanical data, laboratory petrophysical data, mechanical strength data, drilling fluid data, grain information, mineralogy, and field well log data. The method 300 can further include training a neural network to match the known parameters to the one or more scans of the input specimen at 306. The training of the neural network at 306 can be performed by a training module (e.g., the training module 108), a neural network module (e.g., the neural network module 118), or a parameter matcher thereof (e.g., the parameter matcher 112). The neural network can be a convolutional neural network, a modular neural network, an auto-encoder neural network, or any combination thereof. The neural network can be trained at 306 to determine patterns, trends, or correlations between the scans taken at 302 and the known parameters received at 306. In some embodiments, the neural network can be trained at 306 to generate a new correlation between the one or more scans and the known parameters. In further embodiments, however, the neural network can be trained at 306 to refine an existing correlation between the one or more scans and the known parameters to approach a predictive model.

The method 300 can include receiving one or more measurement results of the input specimen at 308. The one or more measurement results (e.g., the measurement results 114) can be used to determine any connection between the scans taken at 302 and results from additional or traditional testing methods. In some embodiments, the one or more measurement results can include results from fluid displacement testing, caliper method testing, and Archimedes principal testing with mercury. The method 300 can further include training the neural network to match the measurement results (e.g., via the result matcher 116) to scans of the input specimen at 310. The matching at 310 can determine patterns, trends, or correlations between the scans taken at 302 and the measurement results received at 308 from further testing on the input specimen. The training performed at 310 can enable prediction of the measurement results from scans alone when used within a fully trained predictive model.

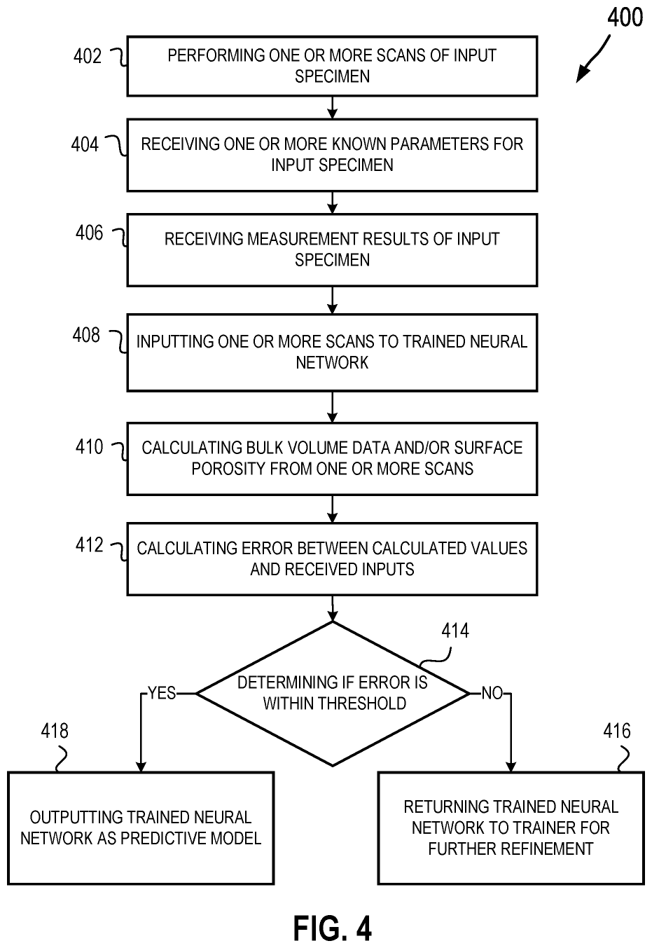

In some embodiments, the method 300 can continue at 302 with receiving one or more scans of a further input specimen, such that further training of the neural network may be performed on a new sample input specimen. The method 300 can continue cyclically training the neural network on known parameters and measurement results until a pre-determined number of training sets have been performed, or until an error threshold is reached (scc FIG. 4). After a desired amount of training has been performed using the method 300, the method 300 can continue at 312 with outputting a trained neural network for assessment or use.

FIG. 4 is an example of a method 400 for testing a neural network on scans of an input specimen with known parameters. The method 400 can be implemented by the system 100 and the scanner assembly 104, as shown in FIGS. 1-2. Further, the method 400 can be implemented as an extension or follow-up to the method 300 for training a neural network. Thus, reference can be made to the example of FIGS. 1-3 in the example of FIG. 4. The method 400 can begin at 402 with performing one or more scans of an input specimen (e.g., the input specimen 102). The one or more scans performed at 402 can be performed by a scanner assembly (e.g., the scanner assembly 104) and can include an x-ray reflective scan, a laser reflective scan, an optical image scan, an acoustic reflective scan, or any combination thereof (e.g., performed by the scanners 104a-d). As above, the scans taken at 402 can further include scans of one or more surface molds for the input specimen, such that surface porosity and features can be captured via the scanner assembly. The method 400 can further include receiving one or more known parameters for an input specimen at 404 and receiving one or more measurement results of the input specimen at 406. The known parameters received at 404 and the measurement results received at 406 can include the same parameters and results discussed at 304 and 308 of FIG. 3.

The method 400 can further include inputting the one or more scans performed at 402 into a trained neural network (e.g., the trained neural network output at 312). The input of the one or more scans to the trained neural network can enable assessment of the trained neural network without providing the known parameters and measurement results thereto. The method 400 can continue at 410 with calculating bulk volume data and/or surface porosity for the input specimen using the one or more scans input at 408. The trained neural network can utilize a bulk volume data calculator (e.g., the bulk volume data calculator 120) and/or a surface pore volume calculator (e.g., the surface pore volume calculator 122) for performance of the calculations and/or generation of parameters and results. The bulk volume data and the surface pore volume data can include one or more of the parameters discussed above, such that any of the trained inputs can be output by the trained neural network for prediction of these values.

The method 400 can continue at 412 with calculating an error between the calculated values determined at 410 and the true values of the known parameters and measurement results received at 404 and 406. The error can be calculated via a decision module (e.g., the decision module 124) and/or an error calculator thereof (e.g., the error calculator 126). The error calculated at 412 can be represented as a mean, median, or maximum error for each desired output for the trained neural network. The method 400 can continue at 414 with determining if the error calculated at 412 is within a pre-determined threshold. In some embodiments, the pre-determined threshold at 414 can be about 5% to provide statistically significant correlations. In further embodiments, however, the pre-determined threshold can be lower to provide greater confidence in the trained neural network. In some embodiments, the error calculated at 412 will be a mean error value with error bars to determine error trends as training continues. The error trend can be utilized to improve the algorithm for the neural network and can aid in creation of further neural network models based on the specific rock fabric found in the input specimen.

If the error is determined to be greater than the threshold value at 414, the method 400 can continue at 416 with returning the trained neural network to the trainer for further refinement of the neural network. In some embodiments, the method 400 can include the method 300, such that the method 400 can continue at 302 with continued training of the neural network as a result of a failing error value at 414. In these embodiments, further training data and test cases can be input to the trainer and neural network to increase accuracy and reliability of the predictive model.

If the error is determined to be less than the threshold value at 414, the method 400 can continue at 418 with outputting the trained neural network as a predictive model. The predictive model (e.g., the predictive model 128) can be output from the system to an operator device, a processing device (e.g., the processing device 130), a cloud-based system, or any other device capable of performing predictive modeling using the trained neural network. The predictive model can be deployed for use in informing operations within a body of interest, such as a hydrocarbon reservoir, with a confidence level defined by the error threshold used at 414.

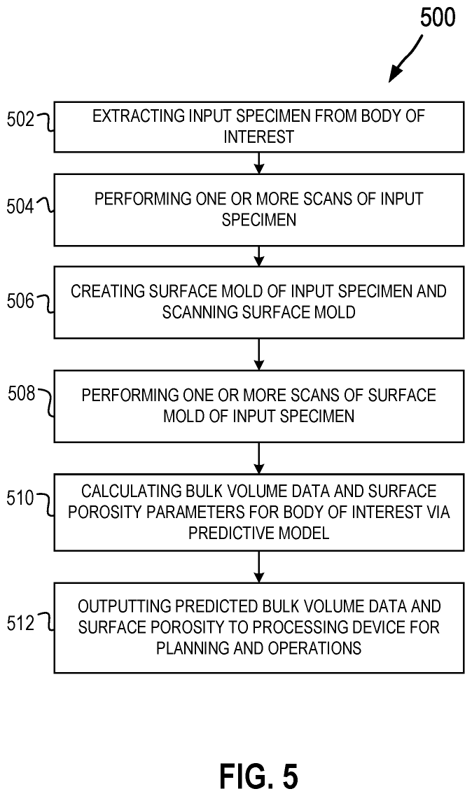

FIG. 5 is an example of a method 500 for deploying a trained neural network as a predictive model for an unknown body of interest. The method 500 can be implemented by the system 100 and the scanner assembly 104, as shown in FIGS. 1-2. Further, the method 500 can be implemented as an extension or follow-up to the methods 300 and 400 for training and testing a neural network. Thus, reference can be made to the example of FIGS. 1-4 in the example of FIG. 5. The method 500 can begin at 502 with extracting an input specimen from a body of interest. In some embodiments, the input specimen can be a cylindrical rock sample removed from a hydrocarbon reservoir, such that the method 500 can utilize the input specimen to predict behavior of the overall hydrocarbon reservoir. In further embodiments, however, the input specimen can be sourced from an agricultural (e.g., partial crop yield), pharmaceutical (e.g., drug manufacturing sample), medical (e.g., tissue sample or bacterial culture), construction (e.g., cement sample), food preparation and packing (e.g., quality checks for packaged foods), or any other application in which bulk volume predictions can be performed using sample specimens.

The method 500 can continue at 504 with performing one or more scans of the input specimen. As discussed above, the scans performed at 504 can include optical imaging, x-ray reflective, acoustic reflective, and/or laser reflective scanning. In some embodiments, the scans performed at 504 may be performed by a single scanner assembly (e.g., the scanner assembly 104 as shown in FIG. 2). In these embodiments the scanner assembly can autonomously switch between scanners, rotate the input specimen, and flip the input specimen to provide a full scan of the input specimen using each included scanner. In some embodiments, the scanners can be utilized in taking multi-layer scans of the input specimen (e.g., via the x-ray and acoustic reflective scanners) in order to assess surface deformation and failure for use in drilling operation planning and mechanical strength estimation.

The method 500 can continue at 506 with creating a surface mold of the input specimen and scanning the generated surface mold. The surface mold can be generated to enable enhanced scanning of the surface features and roughness of the input specimen. The surface mold can enable surface structure scans to be created to aid in prediction of volumes for surface pores, cracks, and fractures, as well as for use in determination of whether these features developed in-situ or post-extraction. As with the input specimen, the method 500 can continue at 508 with performing one or more scans of the surface mold in order to generate the surface scans discussed above. The scans performed at 508 can utilize the same scanners discussed in the scanning of the input assembly, can be selectively scanned by specific scanners, or can utilize additional scanning technology not used on the input specimen.

The method 500 can continue at 510 with calculating bulk volume data and surface porosity parameters for the body of interest via a predictive model. As discussed above, the predictive model can be an output of the method 400, such that a certain confidence level is attained for the predictive model to be used. The predictive model can be trained on a plurality of input specimens and training data, and can yield a number of bulk volume calculations and surface porosity predictions. In hydrocarbon reservoir applications, the predictive model can predict values including, but not limited to, length, diameter, volume, bulk volume, surface porosity, total pore volume, compressibility information, linear stress and strain, mechanical strength data, drilling fluid data, grain information, mineralogy, well log data, and any combination thereof. The accurate prediction of these values using the method 500 can enable increased understandings of porosity and permeability of the hydrocarbon reservoir, which can limit losses due to early well abandonment, low reserve and production estimations, and other hydrocarbon reservoir inaccuracies during operations. Accordingly, the method 500 can continue at 512 with outputting the predicted bulk volume data and surface porosity to an operator device or processing device for use in planning and operations in the body of interest. For example, a drilling operator or well planner can utilize the predicted data to plan and perform well drilling operations with accurate reserve and production capacities.

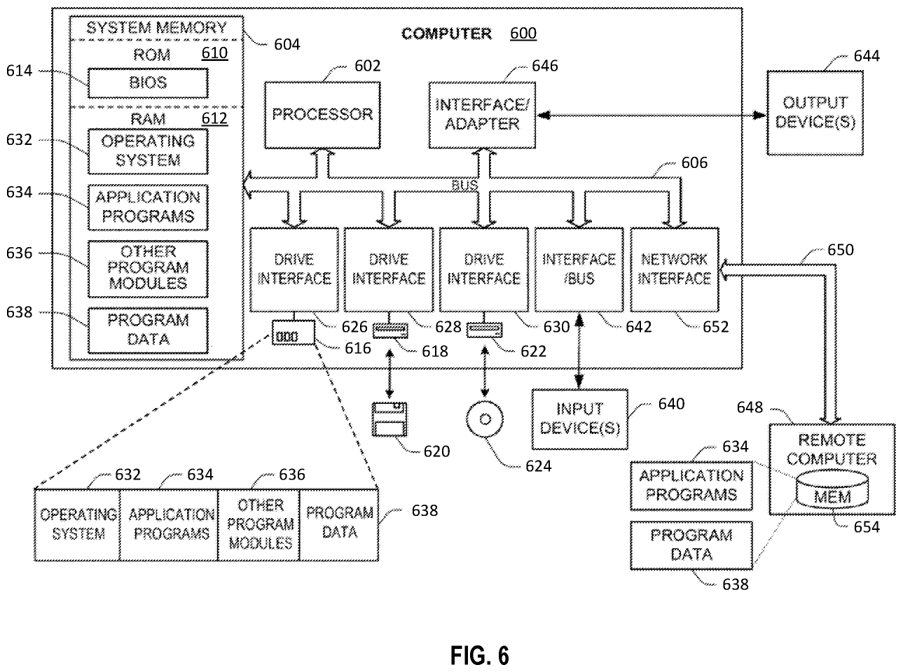

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 6. Furthermore, portions of the embodiments may be a computer program product on a computer-readable storage medium having computer readable program code on the medium. Any non-transitory, tangible storage media possessing structure may be utilized including, but not limited to, static and dynamic storage devices, volatile and non-volatile memories, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to realize a computer implemented process such that the instructions which execute on the computer or other programmable apparatus provide steps for implementing the functions specified in flowchart blocks that may be described herein.

In this regard, FIG. 6 illustrates one example of a computer system 600 that can be employed to execute one or more embodiments of the present disclosure. Computer system 600 can be implemented on one or more general purpose networked computer systems, embedded computer systems, routers, switches, server devices, client devices, various intermediate devices/nodes or standalone computer systems. Additionally, computer system 600 can be implemented on various mobile clients such as, for example, a personal digital assistant (PDA), laptop computer, pager, and the like, provided it includes sufficient processing capabilities.

Computer system 600 includes processing unit 602, system memory 604, and system bus 606 that couples various system components, including the system memory 604, to processing unit 602. System memory 604 can include volatile (e.g. RAM, DRAM, SDRAM, Double Data Rate (DDR) RAM, etc.) and non-volatile (e.g. Flash, NAND, etc.) memory. Dual microprocessors and other multi-processor architectures also can be used as processing unit 602. System bus 606 may be any of several types of bus structure including a memory bus or memory controller, a peripheral bus, and a local bus using any of a variety of bus architectures. System memory 604 includes read only memory (ROM) 610 and random access memory (RAM) 612. A basic input/output system (BIOS) 614 can reside in ROM 610 containing the basic routines that help to transfer information among elements within computer system 600.

Computer system 600 can include a hard disk drive 616, magnetic disk drive 618, e.g., to read from or write to removable disk 620, and an optical disk drive 622, e.g., for reading CD-ROM disk 624 or to read from or write to other optical media. Hard disk drive 616, magnetic disk drive 618, and optical disk drive 622 are connected to system bus 606 by a hard disk drive interface 626, a magnetic disk drive interface 628, and an optical drive interface 630, respectively. The drives and associated computer-readable media provide nonvolatile storage of data, data structures, and computer-executable instructions for computer system 600. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and ROM 610, including operating system 632, one or more application programs 634, other program modules 636, and program data 638. In some examples, the application programs 634 can include the training module 108, the neural network module 118, the decision module, and any sub-programs thereof, and the program data 638 can include known parameters 110, measurement results 114, scans from scanner assembly 104, training data used in training module 108, and errors calculated in error calculator 126. The application programs 634 and program data 638 can include functions and methods programmed to train, test, and utilize a neural network to predict bulk volume and surface porosity characteristics using an input specimen from a body of interest, such as shown and described herein.

A user may enter commands and information into computer system 600 through one or more input devices 640, such as a pointing device (e.g., a mouse, touch screen), keyboard, microphone, joystick, game pad, scanner, and the like. For instance, the user can employ input device 640 to edit or modify scans from the scanner assembly 104, a threshold in the error calculator 126, input known parameters 110 and measurement results 114, as well as any other manual functions of the system 100. These and other input devices 640 are often connected to processing unit 602 through a corresponding port interface 642 that is coupled to the system bus, but may be connected by other interfaces, such as a parallel port, serial port, or universal serial bus (USB). One or more output devices 644 (e.g., display, a monitor, printer, projector, or other type of displaying device) is also connected to system bus 606 via interface 646, such as a video adapter.

Computer system 600 may operate in a networked environment using logical connections to one or more remote computers, such as remote computer 648. Remote computer 648 may be a workstation, computer system, router, peer device, or other common network node, and typically includes many or all the elements described relative to computer system 600. The logical connections, schematically indicated at 650, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 600 can be connected to the local network through a network interface or adapter 652. When used in a WAN networking environment, computer system 600 can include a modem, or can be connected to a communications server on the LAN. The modem, which may be internal or external, can be connected to system bus 606 via an appropriate port interface. In a networked environment, application programs 634 or program data 638 depicted relative to computer system 600, or portions thereof, may be stored in a remote memory storage device 654.

Although this disclosure includes a detailed description on a computing platform and/or computer, implementation of the teachings recited herein are not limited to only such computing platforms. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models (e.g., software as a service (Saas, platform as a service (PaaS), and/or infrastructure as a service (IaaS)) and at least four deployment models (e.g., private cloud, community cloud, public cloud, and/or hybrid cloud). A cloud computing environment can be service oriented with a focus on statelessness, low coupling, modularity, and semantic interoperability.

Embodiments disclosed herein include:

A. A system for generating a predictive model using an input specimen includes a bulk volume prediction engine to generate the predictive model using one or more scans of the input specimen, the bulk volume prediction engine including a training module to train a neural network on training data including scans of training input specimens and known parameters for training input specimens, a neural network module to determine bulk volume data and surface pore volume calculations for a body of interest using the scans of the input specimen as an input to a trained neural network, and a decision module to test results of the trained neural network compared against known training parameters.

B. A computer-implemented method for training a neural network to predict bulk volume data from an input specimen includes performing, via a scanner assembly, one or more scans of an input specimen from a body of interest with known bulk volume parameters, receiving one or more of the known bulk volume parameters for the input specimen or body of interest, and training, via a bulk volume prediction engine, a neural network model to correlate the one or more scans to the known bulk volume parameters through creation of a correlation or refinement of an existing correlation, wherein the one or more scans are selected from the group consisting of an optical image scan, a laser reflective scan, an acoustic reflective scan, an x-ray reflective scan, and any combination thereof.

C. A computer-implemented method for predicting bulk volume measurements of a body of interest via a trained neural network includes performing, via a scanner assembly, one or more scans of an input specimen from the body of interest without known bulk volume parameters, calculating, via a predictive model, bulk volume data and/or surface porosity parameters for the body of interest using the one or more scans of the input specimen, and outputting calculated bulk volume data and/or surface porosity parameters for the body of interest to a processing device, wherein the predictive model includes a neural network model trained on scans of test specimens with known parameters.

Each of embodiments A through C may have one or more of the following additional elements in any combination: Element 1: wherein the training data includes results from measurement techniques performed on the training input specimens, and wherein the results are further calculated within the neural network. Element 2: wherein the training module includes a parameter matcher to correlate the scans of the training input specimens to the known parameters, and wherein the training module further includes a result matcher to correlate scans of the training input specimens to the results from the measurement techniques. Element 3: further comprising: a scanner assembly including one or more scanners and operable to perform the one or more scans of the input specimen, wherein the one or more scans extract each surface of the input specimen. Element 4: wherein the scanner assembly further includes a specimen base rotatably coupled to a specimen motor, and wherein the specimen motor is operable to rotate the specimen base while the input specimen is disposed thereon. Element 5: wherein the scanner assembly further includes a flipper arm at or near the specimen base and operable to flip the input specimen to expose a previously disposed surface of the input specimen. Element 6: wherein the scanner assembly further comprises two or more scanners oriented perpendicularly around a structure, and wherein the structure is rotatable to place each scanner in alignment with the input specimen. Element 7: wherein the two or more scanners are selected from the group consisting of an optical image scanner, an x-ray reflective scanner, an acoustic reflective scanner, a laser reflective scanner, and any combination thereof. Element 8: further comprising: a surface mold formed around the input specimen to extract surface characteristics of the input specimen; and one or more scans of the surface mold provided to the bulk volume prediction engine to calculate surface pore volume.

Element 9: wherein the body of interest is a hydrocarbon reservoir and the input specimen is a rock sample extracted therefrom. Element 10: wherein the predictive model uses one or more scans of the rock sample to determine parameters selected from a group consisting of length, diameter, volume, bulk volume, compressibility, linear stress-strain response, petrophysical data, drilling fluid data, grain information, mineralogy data, and any combination thereof. Element 11: further comprising: creating a surface mold of the input specimen to capture one or more surface features of the input specimen; and scanning, via the scanner assembly, the surface mold of the input specimen to extract the surface features of the input specimen. Element 12: further comprising: performing, via the scanner assembly, one or more scans of a test input specimen with known bulk volume parameters and a test surface mold; generating, via a trained neural network model, a predicted value of one or more of the known bulk volume parameters for the test input specimen using the one or more scans of the test input specimen and test surface mold; calculating an error between the predicted value and the known bulk volume parameters; and comparing the error to a pre-determined threshold to determine if the trained neural network model is ready for deployment. Element 13: further comprising: determining that the error is greater than the pre-determined threshold; performing, via the scanner assembly, one or more scans of a further input specimen from a further body of interest with further known bulk volume parameters; receiving one or more of the further known bulk volume parameters for the input specimen or body of interest; and retraining, via the bulk volume prediction engine, the trained neural network model to refine an existing correlation. Element 14: further comprising: receiving results of one or more measurement techniques performed on the input specimen; and training, via the bulk volume prediction engine, the neural network model to correlate the one or more scans to the results of the one or more measurement techniques. Element 15: further comprising: creating a surface mold of the input specimen to capture one or more surface features of the input specimen; and performing, via the scanner assembly, one or more scans of the surface mold of the input specimen to extract the surface features of the input specimen, wherein calculating the bulk volume data and/or surface porosity parameters for the body of interest further uses the one or more scans of the surface mold. Element 16: wherein the body of interest is a hydrocarbon reservoir and the input specimen is a rock sample extracted therefrom. Element 17: wherein the one or more scans include an optical image scan, a laser reflective scan, an x-ray reflective scan, and an acoustic reflective scan.

By way of non-limiting example, exemplary combinations applicable to A through C include: Element 1 with Element 2; Element 3 with Element 4; Element 4 with Element 5; Element 3 with Element 6; Element 6 with Element 7; Element 9 with Element 10; Element 11 with Element 12; and Element 12 with Element 13.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A system for generating a predictive model using an input specimen, the system comprising:
a bulk volume prediction engine to generate the predictive model using one or more scans of the input specimen, the bulk volume prediction engine including:
a training module to train a neural network on training data including scans of training input specimens and known parameters for training input specimens,
a neural network module to determine bulk volume data and surface pore volume calculations for a body of interest using the scans of the input specimen as an input to a trained neural network, and
a decision module to test results of the trained neural network compared against known training parameters.

2. The system of claim 1, wherein the training data includes results from measurement techniques performed on the training input specimens, and wherein the results are further calculated within the neural network.

3. The system of claim 2, wherein the training module includes a parameter matcher to correlate the scans of the training input specimens to the known parameters, and wherein the training module further includes a result matcher to correlate scans of the training input specimens to the results from the measurement techniques.

4. The system of claim 1, further comprising:
a scanner assembly including one or more scanners and operable to perform the one or more scans of the input specimen,
wherein the one or more scans extract each surface of the input specimen.

5. The system of claim 4, wherein the scanner assembly further includes a specimen base rotatably coupled to a specimen motor, and wherein the specimen motor is operable to rotate the specimen base while the input specimen is disposed thereon.

6. The system of claim 5, wherein the scanner assembly further includes a flipper arm at or near the specimen base and operable to flip the input specimen to expose a previously disposed surface of the input specimen.

7. The system of claim 4, wherein the scanner assembly further comprises two or more scanners oriented perpendicularly around a structure, and wherein the structure is rotatable to place each scanner in alignment with the input specimen.

8. The system of claim 7, wherein the two or more scanners are selected from the group consisting of an optical image scanner, an x-ray reflective scanner, an acoustic reflective scanner, a laser reflective scanner, and any combination thereof.

9. The system of claim 1, further comprising:
a surface mold formed around the input specimen to extract surface characteristics of the input specimen; and
one or more scans of the surface mold provided to the bulk volume prediction engine to calculate surface pore volume.

10. The system of claim 1, wherein the body of interest is a hydrocarbon reservoir and the input specimen is a rock sample extracted therefrom.

11. The system of claim 10, wherein the predictive model uses one or more scans of the rock sample to determine parameters selected from a group consisting of length, diameter, volume, bulk volume, compressibility, linear stress-strain response, petrophysical data, drilling fluid data, grain information, mineralogy data, and any combination thereof.

12. A computer-implemented method for training a neural network to predict bulk volume data from an input specimen, the method comprising:
performing, via a scanner assembly, one or more scans of an input specimen from a body of interest with known bulk volume parameters;
receiving one or more of the known bulk volume parameters for the input specimen or body of interest; and
training, via a bulk volume prediction engine, a neural network model to correlate the one or more scans to the known bulk volume parameters through creation of a correlation or refinement of an existing correlation,
wherein the one or more scans are selected from the group consisting of an optical image scan, a laser reflective scan, an acoustic reflective scan, an x-ray reflective scan, and any combination thereof.

13. The computer-implemented method of claim 12, further comprising:
creating a surface mold of the input specimen to capture one or more surface features of the input specimen; and
scanning, via the scanner assembly, the surface mold of the input specimen to extract the surface features of the input specimen.

14. The computer-implemented method of claim 13, further comprising:
performing, via the scanner assembly, one or more scans of a test input specimen with known bulk volume parameters and a test surface mold;
generating, via a trained neural network model, a predicted value of one or more of the known bulk volume parameters for the test input specimen using the one or more scans of the test input specimen and test surface mold;
calculating an error between the predicted value and the known bulk volume parameters; and
comparing the error to a pre-determined threshold to determine if the trained neural network model is ready for deployment.

15. The computer-implemented method of claim 14, further comprising:
determining that the error is greater than the pre-determined threshold;
performing, via the scanner assembly, one or more scans of a further input specimen from a further body of interest with further known bulk volume parameters;
receiving one or more of the further known bulk volume parameters for the input specimen or body of interest; and
retraining, via the bulk volume prediction engine, the trained neural network model to refine an existing correlation.

16. The computer-implemented method of claim 12, further comprising:
receiving results of one or more measurement techniques performed on the input specimen; and
training, via the bulk volume prediction engine, the neural network model to correlate the one or more scans to the results of the one or more measurement techniques.

17. A computer-implemented method for predicting bulk volume measurements of a body of interest via a trained neural network, the method comprising:
performing, via a scanner assembly, one or more scans of an input specimen from the body of interest without known bulk volume parameters;
calculating, via a predictive model, bulk volume data and/or surface porosity parameters for the body of interest using the one or more scans of the input specimen; and
outputting calculated bulk volume data and/or surface porosity parameters for the body of interest to a processing device,
wherein the predictive model includes a neural network model trained on scans of test specimens with known parameters.

18. The computer-implemented method of claim 17, further comprising:
creating a surface mold of the input specimen to capture one or more surface features of the input specimen; and
performing, via the scanner assembly, one or more scans of the surface mold of the input specimen to extract the surface features of the input specimen,
wherein calculating the bulk volume data and/or surface porosity parameters for the body of interest further uses the one or more scans of the surface mold.

19. The computer-implemented method of claim 17, wherein the body of interest is a hydrocarbon reservoir and the input specimen is a rock sample extracted therefrom.

20. The computer-implemented method of claim 17, wherein the one or more scans include an optical image scan, a laser reflective scan, an x-ray reflective scan, and an acoustic reflective scan.
